(19) United States
(12) Patent Application Publication
(10) Pub. No.: US 2015/0037882 A1
(43) Pub. Date: Feb. 5, 2015

(54) SCALABLE PROCESS FOR THERAPEUTIC CELL CONCENTRATION AND RESIDUAL CLEARANCE

(71) Applicants: Jon Rowley, Walkersville, MD (US); Jacob Pattasseril, Mt. Airy, MD (US); Lye Theng Lock, Frederick, MD (US)

(72) Inventors: Jon Rowley, Walkersville, MD (US); Jacob Pattasseril, Mt. Airy, MD (US); Lye Theng Lock, Frederick, MD (US)

(73) Assignee: LONZA WALKERSVILLE, INC, Walkersville, MD (US)

(21) Appl. No.: 14/367,428

(22) PCT Filed: Dec. 21, 2012
(86) PCT No.: PCT/US12/71259

§ 371 Date: Jun. 20, 2014

Related U.S. Application Data
(60) Provisional application No. 61/578,362, filed on Dec. 21, 2011.

(51) Int. Cl. C12M 1/00; C12N 5/00

(52) U.S. Cl. CPC C12M 47/02; C12N 5/00
USPC 435/325

(57) ABSTRACT

Apparatus and corresponding method for concentration and washing of live mammalian cells, for preparation of human cell therapy products. Optimized parameters for a temperature regulated, completely closed, fully disposable and scalable counterflow centrifugation separation system having integrated disposables designed for both the input cells and output cells are provided.

### FIELD OF THE INVENTION

The present invention relates to a volume reduction and wash technology for cell therapy. More particularly, this invention relates to concentrating and washing mammalian cells using counterflow centrifugation separation technology, particularly live mammalian cells that are used in therapeutic products.

### BACKGROUND OF THE INVENTION

The Food and Drug Administration (FDA) defines cell therapy as the prevention, treatment, cure or mitigation of disease or injuries in humans by the administration of autologous, allogeneic or xenogeneic cells that have been manipulated or altered ex vivo. The goal of cell therapy, overlapping that of regenerative medicine, is to repair, replace or restore damaged tissues or organs.

Ex vivo expansion of cells obtained from human donors is being used, for example, to increase the numbers of stem and progenitor cells available for autologous and allogeneic cell therapy. For instance, multipotent mesenchymal stromal cells (MSCs) are currently exploited in numerous clinical trials to investigate their potential in immune regulation, hematopoiesis, and tissue regeneration. The low frequency of MSCs necessitates cell expansion to achieve transplantable numbers.

The challenge for any cell therapy is to assure safe and high-quality cells for transplantation, at a reasonable cost and at lot sizes able to support a commercial therapeutic product. In particular, cell processing under current Good Manufacturing Practice (cGMP)-graded conditions is mandatory for the progress of such advanced cell therapies. For allogeneic therapies, the economics of testing and certification of processes and products for cGMP compliance are a significant cost factor in cell manufacturing, strongly encouraging production of maximum batch size and minimum batch run. Importantly, cell therapies must achieve lot sizes that will supply sufficient material to meet commercial demand. Today's lot sizes of 5-20 billion cells per lot are insufficient to produce a commercial product, and lot sizes must increase to the 100 s of billions of cells yielding process volumes of 100-300 liters of cells for downstream processing. Due to inherently expensive manufacturing processes, traditional biopharmaceutical process yields of 50 percent to 70 percent are unacceptable for cell therapy products. The process economics demand lot sizes of greater than 50 billion cells and product recovery well over 80 percent if cell therapies are to be cost competitive with less complex therapeutic products such as small molecules or therapeutic proteins.

Optimally therefore, therapeutic cell manufacturing for clinical-scale expansion would be conducted in a completely automated, closed process from tissue collection through post-culture processing. Such a closed process would facilitate cGMP-compliant manufacturing of cell therapy products in a form suitable for storage and ready for use in a clinical setting, with minimal risk of microbial contamination. Some systems for such closed processes have been developed for relatively small-scale production of autologous cell therapy products (see, e.g., U.S. Pat. App. Pub. No. 2008/0175825 by Hampson et al.), but for various reasons such systems are not readily scaled for larger preparations.

Large-scale automated, closed processes for use of mammalian cells to manufacture proteins, such as biotherapeutics, are well established. However, most such processes are designed to recover a protein product and discard the cells under conditions leading to cell death, either intentionally, as when cells are disrupted for release of intracellular products, or incidentally, when cells are separated from secreted products by harsh methods such as high shear centrifugation or filtration methods. In contrast, processing of therapeutic cells after expansion typically requires cell harvesting, volume reduction, washing, formulation, filling of storage containers and, often, cryopreservation of the product cells, all under conditions maintaining cell viability, biological functionalities, safety and, ultimately, clinical efficacy.

In addition, therapeutic cells are known not to survive processes for handling cells used for protein production due to high mechanical stresses of these techniques and because the cell lines used in protein production typically represent highly-manipulated cell lines which, during extensive replication in culture, may have undergone selection for less sensitivity to mechanical shear forces and physiological stresses than exhibited, for instance, by progenitor or stem cells used in cell therapies. Thus, to retain efficacy, therapeutic cells typically are minimally cultured so as to maintain the original parental phenotype displayed upon isolation from human tissue; and hence, therapeutic cells generally are not selected or genetically engineered to facilitate downstream processing.

As technologies are developed to scale the cell culture processes, the technology required for downstream processing has quickly been overwhelmed. Specifically, volume reduction and washing of large amounts (e.g., 10-100 liters) of therapeutic cell suspensions with current technologies is time consuming and not scalable. Current technology, such as open centrifugation, may require four to eight hours by five to twenty highly trained technicians using tens to hundreds of individual processing vessels, thus increasing manipulations and risk of contamination. Much of the field of cell therapy utilizes small scale blood processing equipment, which cannot be scaled to more than about ten liters per process. Thus, processing time and labor, and production costs are major constraints to be resolved in therapeutic cell volume reduction and washing, and there are further benefits to process equipment that can scale from the five to ten liter range to several hundred liters, while at the same time maintaining the critical quality parameters of the process and resulting cell product. Such critical quality parameters include: cell suspension densities sufficient for therapeutic formulations (e.g., greater than ten million cells/ml in most cases, and at least 30-70 million cells/ml in some cases): high viability of the final cell product to maintain functionality and safety: high yield of cells to minimize loss of the high value cells; and reduction of residual levels of harvest reagents (e.g., trypsin or other enzyme) and media components (e.g., serum components, active growth factors, and the like) to acceptable levels for regulatory purposes.

Accordingly, there is a need for improved processes for manufacturing therapeutic cells, from cell collection through post-culture processing, including processes for efficient volume reduction and washing of cell suspensions with high yields of viable cells and low residual levels of culture or processing components that are detrimental to therapeutic use of the cells, particularly such processes that facilitate manufacturing in automated, closed systems.

The expansion and recovery of therapeutic cells in scalable culture system therefore requires the use of a well-regulated process that minimizes the risk of contamination, prevents product degradation and maintains product functionality while delivering cells at high concentrations and high purity for ease and efficiency during product processing. High purity cell products are important because they consist of human cells that are intended for implantation, transplantation or infusion into a human patient that must meet specific criteria to be used as therapeutics. A typical manufacturing process for cell-based therapy involves production of large scale cells, which are further recovered with high viability, high purity and of high concentration for cryopreservation in high doses before delivery to end users. Typically high viability means greater than 90 percent viable cells at this stage; however, greater than 80 percent is seen as acceptable. High purity is generally considered less than one ppm process residuals as guided by the Code of Federal Regulations (21 CFR §610.15(b)). The challenges of therapeutic cells vs proteins and related difficulties in scale-up are further outlined in Brandenberger et al., Cell Therapy Bioprocessing Integrating Process and Product Development for the Next Generation of Biotherapeutics, BioProcess International, March 2011: 31-37; and Rowley J A, Developing Cell Therapy Biomanufacturing Processes, Chem. Eng. Progr. (SBE Stem Cell Engineering Supplement) November 2010: 50-55.

Thus, one of the main challenges in cell bioprocess technology is to manufacture and process large number of cells to satisfy the demand for lot sizes of up to 5000 doses per lot, with doses ranging from 20 million to 1 billion cells per dose. This necessitates lot sizes of 20 billion cells for low dose products to up to several trillion cells per lot for high dose indications. As cell bioprocesses have a formulation stage where formulation buffers are used to dilute cells to specific dose concentrations in the presence of biopreservative reagents (such as DMSO), cells must be at greater than final concentration prior to the formulation densities, requiring in-process cell concentrations of 0.5-2 fold above final concentrations. These specifications require a downstream technology that is able to concentrate cells up to 10-80 million cells/ml. While it is possible to further concentrate cells after separation, it is not desirable as each additional processing step leads to 5-15 percent loss of cells. If one could achieve high cell concentrations directly post-separation, overall process recoveries would be much higher, thereby achieving greater process economics.

Counterflow centrifugation separation technology is now available such as kSep® commercialized by kSep Systems Corporation. This device provides counterflow centrifugation for the concentration and washing of therapeutic cells. Counter flow centrifugation has been around since the 1940s, and is used in commercial devices such as Elutra, (sold by Caridian BCT) used in cell processing. Exemplary patents related to this technology include U.S. Pat. Nos. 5,622,819; 5,821,116; 6,133,019; 6,703,219; 6,916,652; 7,549,956; 6,214,617; 5,674,173; 4,939,087; US20110207225 and US20110207222. Counterflow centrifugation separation technology such as kSep® operates continuously and retains heavier/denser materials such as cells, while removing supernatant by net force balance from centrifugation and fluid flowrate. The cells remain in suspension during the process. Advantages include low cell shear stress and continuous supply of oxygen and nutrient rich cell suspension which keep the cells nurtured throughout the process.

However, the cell recovery for these systems is about 78 percent at approximately 60 ml/minute normal (processing) flow rate. Lowering the normal flow can increase cell recovery. Problematically, processing at lower flow rates increases the processing time to complete a harvest of about 30 liters to greater than six hours.

There is a need for a system that can process large volume batches in a reasonable time with high recovery, concentration, and product quality. There is a further need for a system that is temperature regulated, completely closed, fully disposable and scalable and includes integrated disposables designed for both the input cells and output cells (capturing waste media and processing buffer, collecting cells, and taking cells into the next processing steps). There is a further need for a system that can process (separate, clarify, recover and collect cells from the fluid media) 20-120 liters of harvested cells in less than six hours, and in alternative embodiments in less than four hours, and routinely recover greater than 85 percent of cells processed, all while maintaining high cell viability (greater than 85 percent), purity (less than 1 ppm bovine serum albumin (BSA)) and cell functionality.

### SUMMARY OF THE INVENTION

The present invention provides process and apparatus for aseptically concentrating and washing live mammalian cells using counterflow centrifugation separation technology. The invention is particularly useful for live mammalian cells that are used in a therapeutic product, such as for volume reduction and washing of suspensions of such cells for formulation for cryopreservation or for administration to a subject. The present invention provides process parameters for a scalable, high yielding post-harvest process for the concentration and washing of 10 s to 100 s of liters of therapeutic cells that maintain quality parameters for cell therapy drugs, and yields a product that does not require further concentration prior to formulation.

It is therefore an object of the invention to provide a post washing process having a greater than 85 percent cell recovery yield.

It is a further object of the invention to provide a post washing process yielding cells having greater than 90 percent viability.

It is a further object of the invention to provide a post washing process yielding residuals of less than one ppm.

It is a further object of the invention to yield a product having a cell concentration of between 10-60 million cells/ml.

It is a further object of the invention to provide a single use, disposable system for streamlined processing and to enable aseptic cell processing.

It is a further object of the invention to provide an optimized initial and final processing flowrate, speed and time of the ramping-up, total cells processed per chamber, processing temperature, total number of cells processed, number of wash volumes, harvest flow rate and total harvest volume, all with a focus on maximizing process yields.

It is a further object of the invention to provide a system and method for therapeutic cell concentration and residual clearance having a starting flowrate of 30-60 ml/minute.

It is a further object of the invention to provide a system and method for therapeutic cell concentration and residual clearance having a processing flowrate of 70-155 ml/minute.

It is a further object of the invention to provide a system and method for therapeutic cell concentration and residual clearance having a harvest flowrate of 50-250 ml/minute.

It is a further object of the invention to provide a system and method for therapeutic cell concentration and residual clearance having a flowrate ramp up time of 3-150 minute.

It is a further object of the invention to provide a system and method for therapeutic cell concentration and residual clearance having a centrifuge speed of 500-1000 rcf (relative centrifugal force).

It is a further object of the invention to provide a system and method for therapeutic cell concentration and residual clearance having an initial dump volume of 45-110 ml.

It is a further object of the invention to provide a system and method for therapeutic cell concentration and residual clearance having a harvest volume of 100-500 ml.

It is a further object of the invention to provide a system and method for therapeutic cell concentration and residual clearance having a wash (volume exchange) of 8-15. In alternative embodiments, it is a further object to provide a system and method for therapeutic cell concentration and residual clearance having a wash (volume exchange) of at least six.

It is a further object of the invention to provide a system and method for therapeutic cell concentration and residual clearance having a temperature of less than 37° C.

It is a further object of the invention to provide a system and method for therapeutic cell concentration and residual clearance having 2-7.5 billion cells per chamber.

It is a further object of the invention to provide a system and method to concentrate cells in a temperature-regulated system.

It is a further object of the invention to provide a system and method for therapeutic cell concentration and residual clearance that can be completed in less than six hours.

It is a further object of the invention to provide a system and method for therapeutic cell concentration and residual clearance that inhibits detrimental effect and/or degradation of cells that may affect the product quality.

It is a further object of the invention to provide an automated sensing/feedback device incorporated into the outcoming cells that monitors in real time viable cell concentration and calculates a final harvest volume to obtain a desired cell concentration.

It is a further object of the invention to provide a closed counterflow centrifugation separation system having single use/disposable components.

It is a further object of the invention to provide a skid based system for therapeutic cell concentration and residual clearance.

It is a further object of the invention to provide a system and method for therapeutic cell concentration and residual clearance having incorporated inline temperature sensor(s) for temperature monitoring.

It is a further object of the invention to provide a system and method for therapeutic cell concentration and residual clearance having incorporated viable cell density sensors with calculation to obtain accurate concentration of harvested cells.

These and other objects are achieved in the present invention. There has thus been outlined, rather broadly, exemplary features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be had to the accompanying drawings and descriptive matter which illustrate preferred embodiments of the invention.

### BRIEF DESCRIPTION OF THE FIGURES

FIG. 1 illustrates a process of the present invention.

FIG. 2 illustrates exemplary operation and control parameters of the present invention.

FIG. 3 illustrates dependency of flow rate to cell recovery.

FIGS. 4A and 4B compares cells recovery, viability and processing time in a fixed vs. ramp-up process.

FIGS. 5A and 5B illustrate ramp-up and fixed fluid flow-rate.

FIG. 6 provides a means for a user to identify the concentration of cells that can be achieved as well as possible cell recovery at different harvest volumes.

FIG. 7 illustrates the dependency of harvest volume to the number of cells processed.

FIG. 8 illustrates effect of number of washes on concentration of BSA in final cell product.

FIG. 9 illustrates the dependency of cell recover on number of cells per container.

FIG. 10 illustrates the dependency of the amount of cells discarded into waster on number of cells processed.

FIG. 11 provides an exemplary embodiment of a closed kSep® style system.

FIG. 12 illustrates typical results using the process of the present invention.

### DETAILED DESCRIPTION OF THE INVENTION

The present invention provides improved methods, and associated apparatus and systems for concentration and washing of mammalian cells, particularly for preparation of live human cell therapy products. Provided herein is a means to address the challenge of processing large volume batches in a reasonable time with high yield and product quality. The disclosed method and system provides optimized parameters for a temperature regulated, completely closed, fully disposable and scalable counterflow centrifugation separation system having integrated disposables designed for both the input cells (cells entering the system) and output cells (capturing waste media and processing buffer, collecting cells, and taking cells into the next processing steps). This system can process (separate, clarify, recover and collect cells from the fluid media) 20-120 liters of harvested cells in less than four to six hours, and routinely recovers over 85 percent of cells processed, all while maintaining high cell viability (greater than 85 percent), purity (less than 1 ppm BSA) and cell functionality. This process has been tested and proven successful in laboratory for processing up to 25 liters harvest volume and recovery of 25 billion cells.

There is therefore disclosed herein an aseptic, single use cell processing technology for cell therapy to achieve cell concentration with high cell viability/recovery of at least 10 million/ml, and in alternate embodiments greater than 20 million/ml. This cell processing technology has a processing time of less than six hours for 10-120 liters, and in alternate embodiments less than four hours. Thorough washing of at least six to ten volume exchange leads to residual BSA levels of less than 1000 ng/ml, and in alternate embodiments less than 200 ng/ml. Cell recovery using the method of the present invention is at least 80 percent, and in alternate embodiments at least 85 percent, and in further alternate embodiments at least 90 percent. As disclosed herein process temperature does not exceed 37° C., in alternate embodiments this temperature does not exceed 28° C. to maintain cell viability. While chambers are reported to hold up to 10 billion cells, data shows that a maximum of seven to eight billion cells per chamber provide for maximum cell recovery. The volume required to harvest cells from each chamber (harvest volume) is calculated as y=37.865x+10 to achieve maximal cell concentration and maximal cell recovery, where y is the harvest volume in mls and x is the number of cells to be processed in billions.

There is further disclosed herein a single use process and device using counterflow centrifugation separation technology that concentrates therapeutic cells by greater than 10-300 fold while maintaining cell viability greater than 90 percent (or viability drop less than ten percent, preferably less than five percent); substantially reduces the residual levels of BSA, harvest reagent, and other culture media components to levels that are acceptable for human administration; and decreases soluble component levels in media by greater than 100 fold, greater than 1000 fold, and even greater than 5-10,000 fold. This process uses slow flowrate ramp-up process at less than two ml/minute through the first 15-90 minutes of the process, or at least 15 minutes to maximize recovery. The process of the present invention provides cells in a solution suitable as formulation for hypothermic or cryopreserved storage and subsequent human administration. These cells can be derived from human tissue including but not limited to bone marrow, placenta, adipose tissue, and genetically modified cells. This system and process are transferable to other mammalian cell systems including primary animal cells and cell lines for veterinary applications, as well as xeno-transplantation therapies. All cell lines are contemplated as long as the cell product is intended for therapeutic applications, or where high purity, high cell concentration, and high viability are required.

There is further disclosed herein a process using counterflow centrifugation separation that reduces the volume of a therapeutic cell suspension while maintaining cell viability between 80-100 percent and providing at least a 50-100 percent yield, and in alternative embodiments at least 85 percent yield, or at least 90 percent yield, where at least ten liters of cells that have been harvested from culture and intended for administration into a patient are volume reduced at least two-fold. This process yields a therapeutic cell composition that has been concentrated at least two-fold, five-fold, ten-fold, 50-fold, 100-fold, or 300-fold. This process also yields a therapeutic cell composition that has been volume reduced using counterflow centrifugation separation after residual BSA, harvest reagents, and culture components have been reduced to less than one ppm. In alternative embodiments these therapeutic cell compositions have been concentrated to achieve at least 10 million/ml, 20 million/ml, 30 million/ml, 40 million/ml, and 50 million/ml with viability at least 80 percent and yields at least 50-80 percent. In some embodiments of the present invention, cells produce less than 2-fold decrease in ATP production. In other embodiments of the present invention, cells produce no more than 20 milliunits per milliliter (mU/ml) lactate dehydrogenase (LDH) per hr per 106 cells or less than 3-fold increase in other shear-induced molecule release.

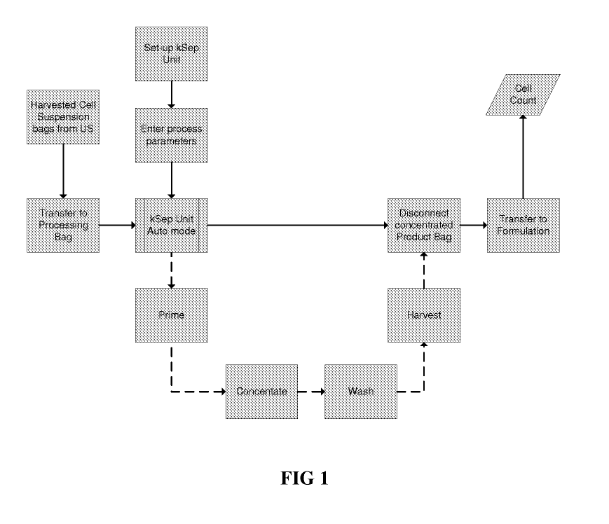

The processing steps for the system of the present invention include: 1/attach/sterile-weld disposable chamber sets and tubing set; 2/ attach tubings to the chamber and media pump; 3/ attach/sterile-weld processing bag to the tubing set's inlet line; 4/attach/sterile-weld waste bag to the tubing set's outlet line; 5/ attach/sterile-weld harvest bag to the tubing set's harvest line; 6/ attach/sterile-weld bag with harvested cells from upstream to the processing bag system; 7/ program system for desired processing parameters; 8/ system priming at low centrifugation; 9/ centrifuge ramp-up; 10/ fluid flow ramp-up from starting to processing flowrate; 11/ switch to washing step; 12/ product harvest; and 13/ quick-disconnect for further cryopreservation procedure. Process flow is further illustrated in FIG. 1.

In one embodiment, the processing steps for the system of the present invention include a disposable manifold unit which is pre-sterilized and adapted for single-time usage, which further includes: (1) tubing having at least one inlet and out outlet end portion, an outside and inside surface, with the inside surface pre-sterilized for passage of a biotechnology fluid flow; (2) plurality of single-use bags, each having access port, one said single-use bag is a buffer bag, one said single-use bag is a processing bag, one said single-use bag is a harvest bag, one said single-use bag is a collection bag, one said single-use bag is a waste bag; (3) a processing bag for passage of cell suspension from harvest bag into elutriation chamber for removing air bubbles and eliminate risk of failure; (4) a collection manifold comprising of 2 bag with pinch valve at a discrete location, said collection of cell product at high concentration in one bag and lower cell concentration in the other bag; and (5) an aseptic disconnector means for operatively disconnecting said length of tubing and collection bag.

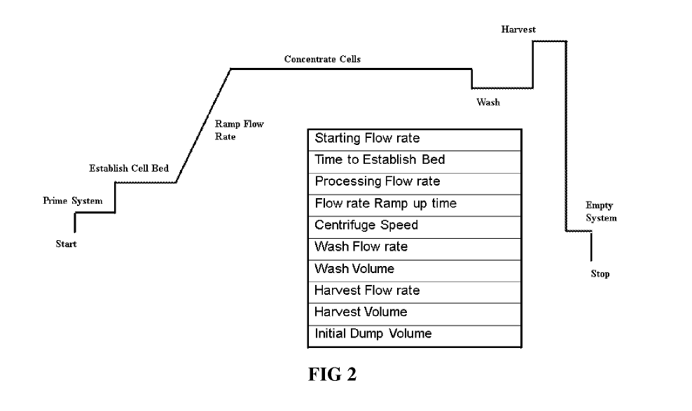

FIG. 2 illustrates operation and control parameters. Exemplary operating parameters and their potential influence on process/performance are provided in Table 1.

TABLE 1

| Exemplary operating parameters | Value | Process Performance |
|---|---|---|
| Starting Flowrate (ml/min) | 30-60 | Cell recovery |
| Processing Flowrate (ml/min) | 70-155 | Cell recovery, viability |
| Harvest Flowrate (ml/min) | 50-250 | Cell recovery, viability |
| Flowrate Ramp up time (min) | 3-150 | Cell recovery, viability |
| Centrifuge Speed (rcf) | 500-1000 | Cell recovery, viability |
| Initial Dump Volume (ml) | 15-35 | Final cell concentration, cell recovery |
| Harvest Volume (ml) | 100-500 | Final cell concentration, cell recovery |
| Wash (Volume Exchange) | 8-15 | Residual BSA in final cells, cell viability |
| Temperature (° C.) | <37 | Cell viability |
| Cells processed per chamber | 2-7.5 billion | Cell recovery |

Starting Flowrate (ml/min)

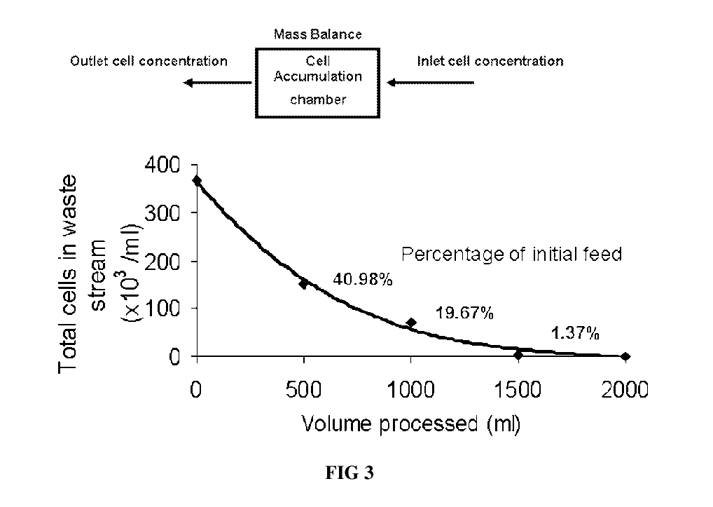

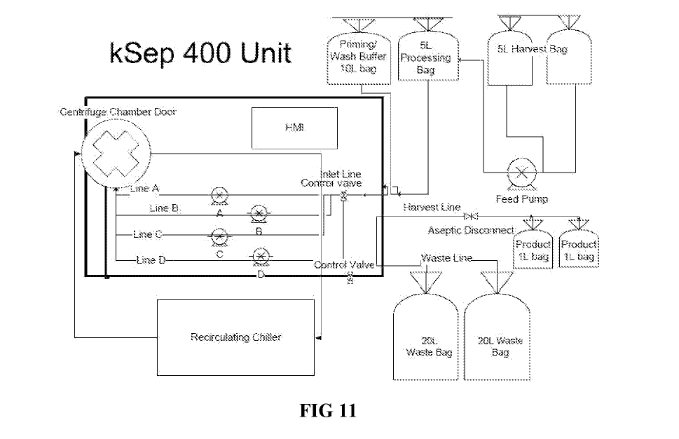

FIG. 3 illustrates the amount of cells lost in waste stream during the initial process, indicating the importance of optimum initial flow rate (or starting flow rate) on cell recovery. For this study, human Dermal Fibroblast (hDF) cells were grown in 40 layer Cell Factories (40 layer CF) in 10 percent FBS/DMEM medium for 8-12 days until they reach confluency. Cells were harvested with 6.4 liters of trypsin and quenched with equal volume of medium before collected in a harvest bag. Ten ml of cell samples were collected with 30-ml sampling syringe. Samples were collected and analyzed with Nucleocounter for cell concentration and viability. Table 2 below lists the experimental conditions of a representative small-scale kSep® run. For this study the kSep® system was set up similar to the set up shown in FIG. 11. Difference in cell sizes, weight or density can directly affect the amount of force required to retain the cells in processing chambers. Cell recovery is optimized by varying the initial and normal processing flowrate while keeping constant centrifugal force at 1000 ref. For example using a fixed processing flowrate of 100 ml/minute, results in greater than 15 percent of cells lost during the separation process. This product loss can be dramatically reduced by decreasing the flowrate at the beginning of the process; that is, the initial or starting flow rate and implementing a ramp-up process at 1-2 ml/minute until a maximum of 150 ml/minute flowrate is achieved. In one embodiment of the present invention the starting flowrate is 30-60 ml/minute.

TABLE 2

| Conditions | Value |
|---|---|
| Initial viable cell density (cells/ml) | 2.29 × 10⁵ |
| Initial cell viability (%) | 96.6 |
| Total volume of cell suspension (L) | 9.94 |
| Total cells | 2.3 × 10⁹ |
| Inlet Flow Rate | 70 |
| Temperature (° C.) | <25 |
| Wash Buffer | plasmAlyte |
| Wash buffer volume (ml) | 1200 |

Processing Flowrate (ml/min)

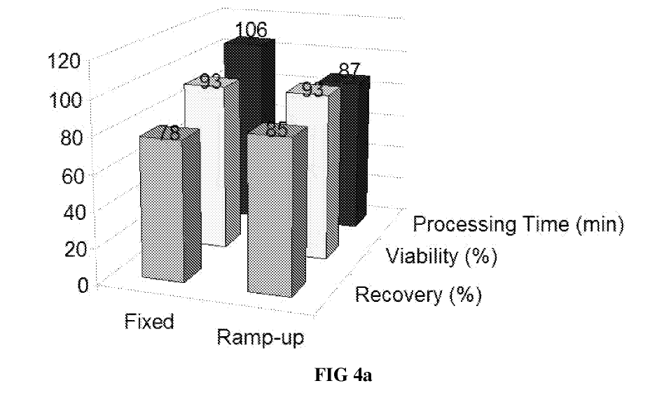

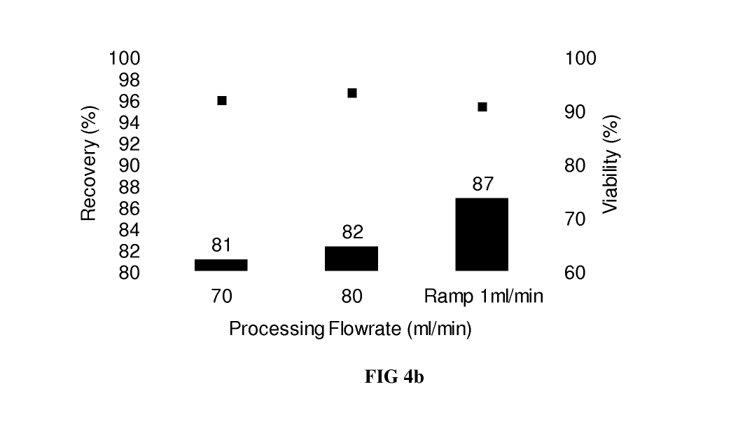

FIGS. 4a and 4b provides comparison of cell recovery, viability and the processing time in a fixed vs. ramp-up process, showing an improved performance in cell recovery and processing time for the ramp-up system. For this study, HDFs and hMSCs were grown in 40LCFs in ten percent FBS/DMEM medium for 8-12 days until they reach confluence. Cells were harvested with 6.4 liters of trypsin and quenched with equal volume of medium before collected in a harvest bag. Ten ml of cell samples were collected with 30 ml sampling syringe. Samples were collected and analyzed with Nucleocounter for cell concentration and viability. Table 3 below lists the experimental conditions of a representative small-scale kSep® run. For this study the kSep® system was set up similar to the set up shown in FIG. 11.

TABLE 3

| Conditions | Value |
|---|---|
| Cell Type | HDF |
| Initial viable cell density (cells/ml) | 2.75 × 10⁵ |
| Initial cell viability (%) | 98.2 |
| Total volume of cell suspension (L) | 3.2 |
| Total cells | 5.79 × 10⁹ |
| Inlet Flow Rate | 120 |
| Temperature (° C.) | <25 |
| Wash Buffer | plasmAlyte |
| Wash buffer volume (ml) | 1000 |

As illustrated by FIG. 4, and as mentioned above, the fixed processing flowrate allows approximately 78 percent of cell recovery for a three billion cell process, while a ten percent total improvement in cell recovery (from 78 percent to 85 percent) was obtained if a ramp-up processing flowrate was implemented. In this ramp-up study, the processing time was reduced by 20 minutes, while final cell viabilities were similar at 93 percent. The critical parameters in the ramp-up process are the rate of the fluid ramp-up as well as the starting and final processing flowrate. In one embodiment of the present invention the processing flowrate is 70-155 ml/minute.

Flowrate Ramp up time (min)

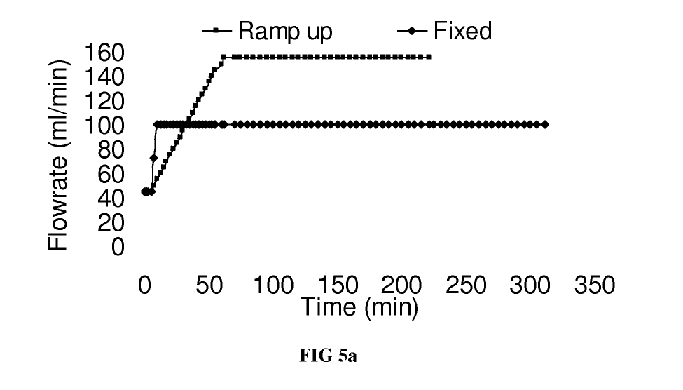

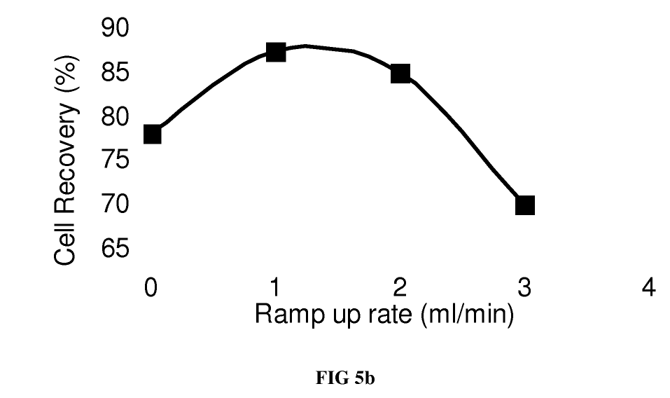

FIG. 5a illustrates modeling of ramp-up (two ml/minute) and fixed fluid flowrate regimen. Here, Chinese Hamster Ovary (CHO) cells were cultivated in spinner or shake flask with PowerCHO serum-free medium supplemented with L-glutamine at 37.0±1° C. and 5.0±1 percent CO₂ for seven to ten days. Cells were collected in two liter roller bottles or five liter harvest bag for kSep® processing. Cell suspension samples were collected to quantify the initial and final cell concentration and viability. Table 4 below lists the experimental conditions of a representative small-scale kSep® run. For this study the kSep® system was set up similar to the set up shown in FIG. 11.

TABLE 4

| Conditions | Value |
|---|---|
| Cell Type | CHO |
| Initial viable cell density (cells/ml) | 2.25 × 10⁶ |
| Initial cell viability (%) | 94.3 |
| Total volume of cell suspension (L) | 3.6 |
| Total cells | 8.2 × 10⁹ |
| Inlet Flow Rate | 140 |
| Temperature (° C.) | <25 |
| Wash Buffer | plasmAlyte |
| Wash buffer volume (ml) | 1000 |

To estimate a larger scale (120 liter) run, a fixed and ramp-up operating regimen was modeled based on a 30 liter cells suspension/chamber and eight washes (0.8 liter) run. From this model, the amount of time required to process the 30.8 liter volume was decreased from 5.3 hours (fixed) to 3.7 hours (ramp-up). This demonstrates that the optimized ramp-up process can allow for improved cell recovery and product quality, as the cells quality is inversely proportional to the processing time. In one embodiment of the present invention the flow-rate ramp up time is 3-150 minutes. FIG. 5b illustrates differences in cell recovery with respect to the ramp up rate. Optimum ramp-up speed is between one to two ml/minute.

Harvest Flowrate (ml/min)

In one embodiment of the present invention the harvest flowrate is 50-250 ml/minute. Low harvest flowrate less than 50 ml/minute results in poor cell recovery (approximately 5 percent drop in recovery) whereas flowrate higher than 250 ml/minute can causes high shear and reduced cell viability.

Centrifuge Speed (rcf)

In one embodiment of the present invention the centrifuge speed is 500-1000 rcf. 500-1000 rcf centrifuge force creates balance of force for fluid flow in the system for processing cells in less than six hours. This processing time range is ensures high cell viability and cell recovery from the process.

### Initial Dump Volume (ml)

In one embodiment of the present invention the initial dump volume is 15-35 ml. Initial dump volume is based on the hold-up volume in the tubings from the cell chambers to the control valve. The tubing can hold between 45 to 110 ml liquid depending on its length post welding and number of chambers used. The hold-up volume is calculated based on the typical length and diameter of the tubing.

### Harvest Volume (ml)

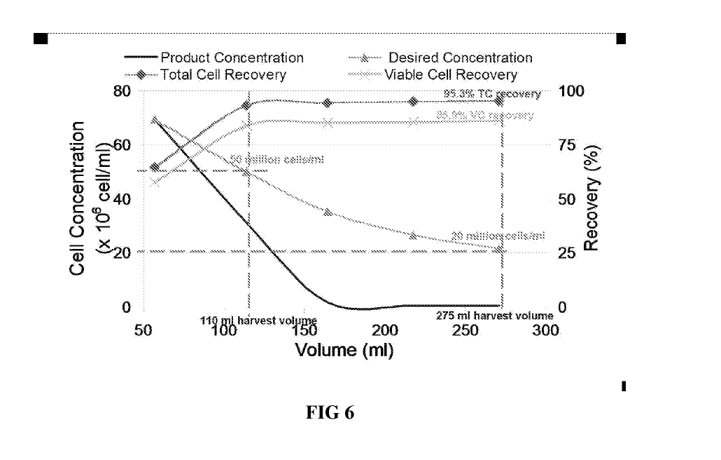

FIG. 6 illustrates data collection during the harvest of a 7.1 billion cell process. This plot allows users to identify the concentrations of cells that can be achieved as well as the cell recoveries at different harvest volume. To identify the critical harvest volume required to achieve the final cell product at high concentration without compromising their recovery, cells harvested from a 7.1 billion cell run were sampled at different intervals and plotted. Here, Chinese Hamster Ovary (CHO) were cultivated in spinner or shake flask with PowerCHO serum-free medium supplemented with L-glutamine at 37.0±1° C. and 5.0±1 percent CO₂ for 7-10 days. Cells were collected in five liter harvest bag for kSep® processing. Cell suspension samples were collected to quantify the initial and final cell concentration and viability. Tables 5a and 5b below lists the experimental conditions and parameters of a representative kSep® run.

TABLE 5a

| Conditions | Value |
|---|---|
| Cell type | CHO |
| Initial viable cell density (cells/ml) | 1-2 × 10⁶ |
| Initial cell viability (%) | 95-99 |
| Total volume of cell suspension (L) | 2-7 |
| Total cells | 7.5-11.0 × 10⁹ |

TABLE 5b

| kSep parameters | Value |
|---|---|
| Starting Flow rate (ml/min) | 30-60 |
| Processing Flow rate (ml/min) | 50-70 |
| Harvest Flow rate (ml/min) | 75-300 |
| Time to Establish Bed (min) | 3 |
| Flow rate Ramp up time (min) | 2-5 |
| Centrifuge Speed (rcf) | 500 |
| Initial Dump Volume (ml) | 15-35 |
| Harvest Volume (ml) | 100-500 |
| Wash Flow rate (ml/min) | 45-65 |
| Wash (Volume Exchange) | 5 |
| Number of chambers used | 1 |

The product/cell concentration (blue line) harvested from the first 110 ml was highly concentrated, greater than 30 million/ml, indicating that the system hold-up volume per chamber is approximately 110 ml. Accordingly, the total cell recovery approaches 92 percent if cells are harvested with 110 ml volume, while the maximum recovery is 95.3 percent. Hence, depending on the requirements of the final product, it is possible to achieve cells at 50 million/ml concentration with 110 ml harvest volume and 92 percent recovery or to obtain 95.3 percent cell recovery at 20 million/ml cell concentration with 275 ml harvest volume. With the hold up volume being approximately 110 ml and the chamber capacity of 7.5 billion cells, the maximum concentration of the final product is approximately 68 million/ml. This study shows that the outcome of the process is highly dependent on the harvest volume, number of cells processed as well as the desired cell recovery and concentration. In one embodiment of the present invention the harvest volume is 100-500 ml.

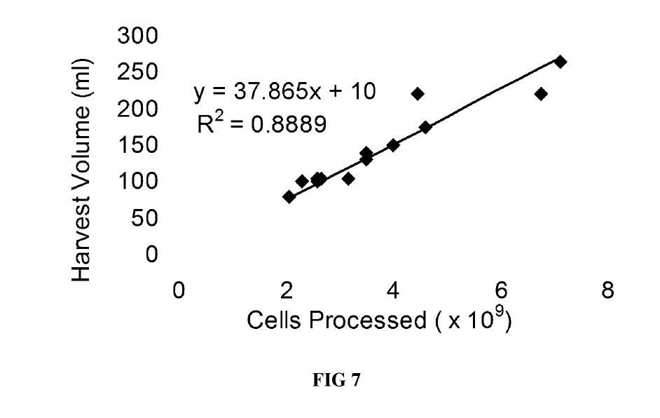

FIG. 7 illustrates that the harvest volume to obtain cells at greater or equal to 20 million/ml is dependent on the number of cells processed. The linear equation allows users to predict the amount of volumes required for their harvest. Subsequently, for the prediction of harvest volume to achieve a typical 20 million cells/ml concentration. Results from multiple studies were compiled to generate the linear equation. For these runs CHO, hMSCs or human dermal fibroblasts (HDFs) were used. The CHO cells were cultivated in spinner or shake flask with PowerCHO serum-free medium supplemented with L-glutamine at 37.0±1° C. and 5.0±1 percent CO₂ for seven to ten days. The hMSC and HDF cells were cultivated in 10 layer or 40 layer cell factories with DMEM/10 percent FBS medium at 37.0±1° C. and 5.0±1 percent CO2 for 10-14 days. Cells were collected in two liter roller bottles or five liter harvest bag for kSep® processing. Cell suspension samples were collected to quantify the initial and final cell concentration and viability. Table 6 below lists the operating parameters used for the kSep® runs.

TABLE 7

| kSep parameters | Value |
|---|---|
| Starting Flowrate (ml/min) | 30-60 |
| Processing Flowrate (ml/min) | 70-155 |
| Harvest Flowrate (ml/min) | 50-250 |
| Time to Establish Bed (min) | 3-5 |
| Flowrate Ramp up time (min) | 3-150 |
| Centrifuge Speed (rcf) | 500-1000 |
| Initial Dump Volume (ml) | 15-35 |
| Harvest Volume (ml) | 100-500 |
| Wash Flowrate (ml/min) | 70-150 |
| Wash (Volume Exchange) | 10-20 |

To obtain 20 million cells/ml concentration with a 7.1 billion cells process, the required harvest volume was 275 ml (FIG. 6). Similarly, a correlation from compiled harvest volumes can be represented by a linear equation y=37.865x+10 for estimation of harvest volume for the kSep® process.

The equation y=37.865x+10 incorporates the percentage of cells recovery as well as the tubing hold-up volume. An automated sensing/feedback device is therefore contemplated and included herein incorporated into the system to monitor the viable cell concentration of the harvest line in real time and calculates the final harvest volume required to achieve the target cell concentration. In one embodiment of the present invention, automation is adapted to the collection step with controls using: a viable cell density sensor means, for monitoring harvest density; a flow meter monitoring means, for monitoring harvest volume; and a control logic software means, to control or achieve the desired final concentration of product cells in collection bag, and for operating valves, wherein said control logic software means are pneumatically or electrically activated and wherein the flow of fluid can be diverted from one collection bag to the other.

### Wash (Volume Exchange)

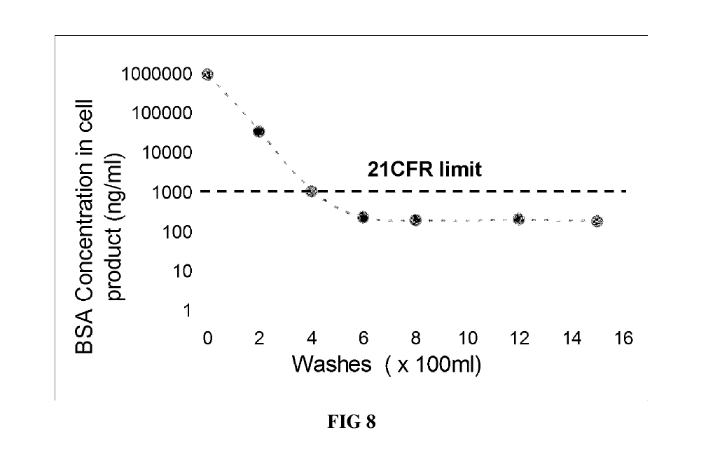

FIG. 8 illustrates the reduction of concentration of BSA in final cell product with increasing number of washes. For this study, 10-15 ml of samples from final cell supernatant were collected and stored in −20° C. BSA concentration in final product with multiple wash volumes (0-15) was measured within ten days of sampling with BSA ELISA Kit (Bethyl Laboratories, Inc.) according to manufacturer's instruction. Samples were pre-diluted prior to assay to achieve concentrations between detectable ranges (0.69-500 ng/ml). Absorbance was measured with SpectraMax plate reader at 450 nm. FIG. 8 illustrates a decrease in BSA concentration to a constant level (−200 ng/ml) after six washes, indicating that the system reaches its limit for residuals removal. At least four volume equivalent washes are required to remove residual BSA below the CFR limit (21 CFR §610.15(b)) while the lowest BSA concentration in the final product (−200 ng/ml) can be attained with six to eight washes. This study was done based on harvested cells containing an initial five percent FBS or 1-2 g/L BSA. In one embodiment of the present invention the wash (volume exchange) is 8-15. Temperature (° C.)

High temperatures result in cell degradation. In one embodiment of the present invention the temperature is less than 37° C. The temperature of the system is regulated with a recirculating chiller to maintain the processing temperature below 37° C. and in alternate embodiments less than 28° C. to minimize degradation in cell quality.

### Cells Per Chamber

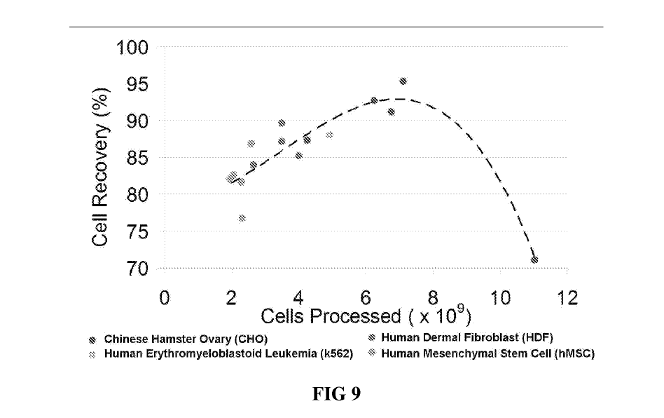

The process performance such as percentage of cell recovery is dominated by the capacity (total cells processed per chamber) of the processing chambers. Capacity of these chambers determines the optimum range of cells number to be processed with the system. FIG. 9 illustrates the number of cells processed per chamber and their corresponding cell recovery. For this study CHO, k562, hMSCs or human dermal fibroblasts (HDFs) were used. The CHO and k562 cells were cultivated in spinner or shake flask with PowerCHO serum-free medium supplemented with L-glutamine or ten percent FBS/DMEM at 37.0±1° C. and 5.0±1 percent CO₂ for seven to ten days. The hMSC and HDF cells were cultivated in 10 layer or 40 layer cell factories with DMEM/10 percent FBS medium at 37.0±1° C. and 5.0±1 percent CO₂ for 10-14 days. Cells were collected in two liter roller bottles or five liter harvest bag for kSep® processing. Cell suspension samples were collected to quantify the initial and final cell concentration and viability. Table 8 below lists the operating parameters used in a representative kSep® run.

TABLE 8

| kSep parameters | Value |
|---|---|
| Starting Flowrate (ml/min) | 30-60 |
| Processing Flowrate (ml/min) | 70-155 |
| Harvest Flowrate (ml/min) | 50-250 |
| Time to Establish Bed (min) | 3-5 |
| Flowrate Ramp up time (min) | 3-150 |
| Centrifuge Speed (rcf) | 500-1000 |
| Initial Dump Volume (ml) | 15-35 |
| Harvest Volume (ml) | 100-500 |
| Wash Flowrate (ml/min) | 70-150 |
| Wash (Volume Exchange) | 10-20 |

As shown in FIG. 9, recovery of cells is dependent on the number of cells being processed per chamber. When less than two billion and when greater than nine billion cells are processed, the percentage of cell recovery drops below 82 percent. The capacity of the system to achieve greater than 85 percent recovery is limited to three to nine billion cells processed per chamber. The optimum recovery is achieved at 95 percent when seven to eight billion cells are processed per chamber. In one embodiment of the present invention 2-7.5 billion cells per chamber are processed.

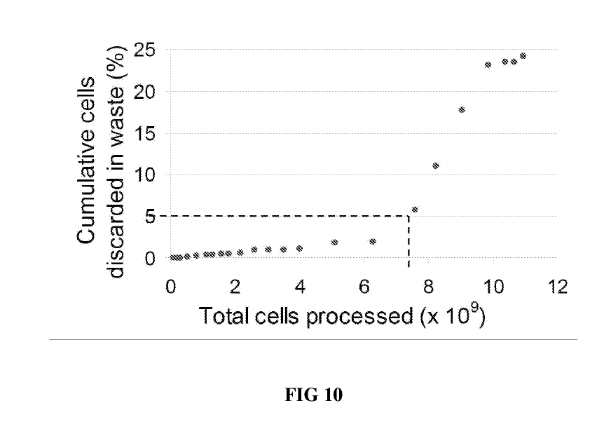

FIG. 10 illustrates the maximum capacity of a standard chamber at which point total cell recovery begins to decrease, and that the amount of cells discarded into waste increases exponentially once the chamber is filled to a specific level. As illustrated, the maximum capacity for a chamber reported to hold up to ten billion cells is seven to eight billion cells to provide for maximum cell recovery. For this study, CHO cells were grown in three liter spinner flask in PowerCHO serum-free medium supplemented with L-glutamine at 37.0±1° C. and 5.0±1 percent CO₂ for seven to ten days. Cells were collected in a ten liter harvest bag. Ten ml of cell samples were collected with 30 ml sampling syringe. Samples were collected from waste stream every five minutes to determine the amount of cells discarded into the waste stream. For this study the kSep® system was set up similar to the set up shown in FIG. 11. Table 9 below lists the operating parameters used in a representative kSep® run.

TABLE 9

| kSep parameters | Value |
|---|---|
| Starting Flowrate (ml/min) | 30-60 |
| Processing Flowrate (ml/min) | 70-155 |
| Harvest Flowrate (ml/min) | 50-250 |
| Time to Establish Bed (min) | 3-5 |
| Flowrate Ramp up time (min) | 3-150 |
| Centrifuge Speed (rcf) | 500-1000 |
| Initial Dump Volume (ml) | 15-35 |
| Harvest Volume (ml) | 100-500 |
| Wash Flowrate (ml/min) | 70-150 |
| Wash (Volume Exchange) | 10-20 |

FIG. 10 illustrates that the decrease in cell recovery when more than 7.5 billion cells are processed is due to cell loss.

### Disposable System

FIG. 11 provides a schematic of a closed counterflow centrifugation separation system set-up. Of importance for the concentration of large scale therapeutic cells is the incorporation of a single-use closed system that allows for aseptic cells processing. A closed system then allows for continuous operation while maintaining products sterility and prevents process failure. Here, the disposable sets are designed with disposable bag which assembles with sterile-welds and/or aseptic quick connects, and disassembles using aseptic quick disconnectors for product recovery to the next cryopreservation process.

At the inlet feed stream, an intermediate processing bag is used to pool cells harvested from culture vessels for feed into the processing chamber(s). This processing bag eliminates the need for intermittent harvest bag exchanges at the inlet feed stream to the kSep unit. Harvest bag exchanges at the inlet feed stream may introduce air bubbles into the processing chamber(s). This design eliminating air bubbles in the processing chamber is important, as accumulation of air bubbles in processing chamber can result in centrifuge imbalance and cause the process to fail. Wash buffer bags have also been designed to incorporate into the system as an aseptic connection.

At the outlet of the harvest stream, there is a system of waste bags to collect the waste media and wash buffer, two cells (product) bags are incorporated for collection of cells harvested from the system. The ability to toggle between product bags allows for collection of cells at different density and further manipulation of cells concentration by dilution within the harvested cells in the bags. The system includes interconnected product bags with aseptic quick-disconnectors for instantaneous detachment from the system. This design is essential to minimize the time between harvests to the next cryopreservation and help preserve the quality of cells between these steps.

### Dead Volume

Cell recovery is lessened and unwanted residuals such as BSA are retained in known systems, due to dead volumes trapped in Y connections of the tubing system. Cell recovery and BSA clearance are improved by reprogramming washes such that they run through all chambers.

### Exemplary Results

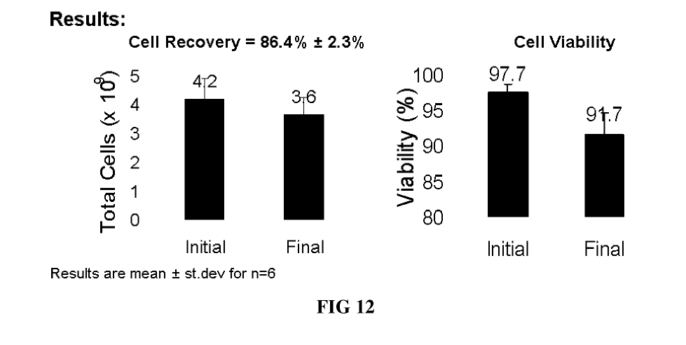

FIG. 12 illustrates typical results using the process of the present invention. In this study human dermal fibroblasts (HDF)/human mesenchymal stem cells were processed in 40 layer cell factories with DMEM/10 percent FBS medium at 37.0±1° C. and 5.0±1 percent CO₂ for 10-14 days before harvesting with six liters of trypsin and six liters of quench solution and collected in a harvest bag for kSep® processing. Ten ml of cell samples were collected with 30 ml sampling syringe. The kSep® system was set up similar to the set up shown in FIG. 11. Table 10 below lists the operating parameters used in the kSep® run.

TABLE 10

| kSep parameters | Value |
|---|---|
| Starting Flowrate (ml/min) | 30-60 |
| Processing Flowrate (ml/min) | 70-155 |
| Harvest Flowrate (ml/min) | 50-250 |
| Time to Establish Bed (min) | 3-5 |
| Flowrate Ramp up time (min) | 3-150 |
| Centrifuge Speed (rcf) | 500-1000 |
| Initial Dump Volume (ml) | 15-35 |
| Harvest Volume (ml) | 100-500 |
| Wash Flowrate (ml/min) | 70-150 |
| Wash (Volume Exchange) | 10-20 |

Known systems have a high risk of entraining air bubbles into the processing chamber. Potential air bubbles would come from during the attachment of multiple harvest bags for a large scale run. In the present invention, as shown in FIG. 11, the risk of air bubbles entering into the chambers is minimized with the use of a Processing Bag. During a large scale run with multiple harvest bags the harvest bags can be attached to the system without the risk of air bubbles entering into the chambers.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention. Each reference cited herein is hereby incorporated in its entirety.

What is claimed is:

1. A method for increasing concentration of a cell suspension and volume reduction, said method comprising the steps of:
introducing the cell suspension into a cell collection chamber of a counter-flow centrifugation apparatus via an inlet at a first flow rate, wherein the direction of the flow of the cell suspension is counter to a centrifugal force generated within the cell collection chamber, wherein the centrifugal force is balanced by the counter flow of the cell suspension at the first flow rate;
introducing a cell collection fluid into the cell collection chamber at the first flow rate for a first predetermined time period to allow cells of the cell suspension to form a cell bed;
modifying the first flow rate of the cell collection fluid to distribute the cells in the cell bed into a plurality of groups of cells within the cell collection chamber based on cell size and density, wherein the group comprising cells having a first size and density is located in close proximity to an exit to the cell collection chamber and the group comprising cells having a second size and density is located in close proximity to the inlet to the cell collection chamber, wherein the first flow rate of the cell collection fluid is modified to follow a first profile, wherein the first profile increases the first flow rate to a second flow rate over a second predetermined time period;
maintaining the cell collection fluid at the second flow rate for a third predetermined time period to increase the concentration of the cells within each of the plurality of groups of cells distributed within the cell collection chamber;
washing the cell collection chamber with a washing fluid for fourth predetermine time period, wherein the washing fluid is introduced into the cell collection chamber at a third flow rate, wherein the washing fluid replaces the cell collection fluid within the cell collection chamber; and
harvesting each of the plurality of groups of cells distributed within the cell collection chamber.

2. The method of claim 1, wherein harvesting each of the plurality of groups of cells distributed within the cell collection chamber further comprises the steps of:
modifying flow rate of the washing liquid to a fourth flow rate; and
collecting the plurality of groups of cells in a plurality of harvest volumes, wherein each of the plurality of harvest volumes comprises a substantial amount of the one of the plurality of groups of cells.

3. The method of claim 2, wherein each of the plurality of harvest volumes is determined using the relationship: y=37.865x+10, wherein y is the harvest volume and x is the number of cells processed.

4. The method of claim 1, further comprising the step of priming the counter-flow centrifugation apparatus with a media fluid.

5. The method of claim 1, further comprising the step of maintaining the cell collection chamber at a temperature of about 37° C.

6. The method of claim 1, wherein the second size is greater than the first size, and wherein the second density is greater than the first density.

7. The method of claim 1, wherein the first flow rate is from about 30 ml/min to about 60 ml/min.

8. The method of claim 1, wherein the first profile increases the first flow rate of the cell collection fluid to the second flow rate of from about 70 ml/min to about 155 ml/min.

9. The method of claim 1, wherein the first profile increases the first flow rate of the cell collection fluid to the second flow rate over the second predetermined time period of from about 3 minutes to about 150 minutes.

10. The method of claim 1, wherein each of the plurality of groups of cells distributed within the cell collection chamber is harvested at the fourth flow rate of from about 50 ml/min to about 250 ml/min.

11. The method of claim 1, wherein the centrifugal force generated within the cell collection chamber has a relative centrifugal force value of from about 500 to about 1000.

12. A method for reducing volume of a cell suspension using counter-flow centrifugation, said method comprising the steps of:
introducing the cell suspension into a cell collection chamber of a counter-flow centrifugation apparatus via an inlet at a first flow rate;
introducing a cell collection fluid into the cell collection chamber at the first flow rate to allow the cells of the cell suspension to form a cell bed;
adjusting the first flow rate of the cell collection fluid to follow a predetermined flow profile to distribute the cells in the cell bed into a plurality of groups of cells within the cell collection chamber, wherein the flow profile increases the first flow rate to a second flow rate over a predetermined time period;
increasing the concentration of the cells within each of the plurality of groups of cells distributed within the cell collection chamber;
washing the cell collection chamber with a predetermined amount of washing fluid, wherein the washing fluid replaces the cell collection fluid within the cell collection chamber; and
harvesting each of the plurality of groups of cells distributed within the cell collection chamber.

13. The method of claim 12, wherein the direction of the flow of the cell suspension is opposite to a centrifugal force generated within the cell collection chamber, wherein the centrifugal force is balanced by the counter flow of the cell suspension at the first flow rate.

14. The method of claim 12, wherein the plurality of groups of cells are distributed within the cell collection chamber based on the cell size and density, wherein the group comprising cells having a first size and density is located in close proximity to an exit to the cell collection chamber and the group comprising cells having a second size and density is located in close proximity to the inlet to the cell collection chamber, wherein the second size and density is greater than the first size and density, wherein the first flow rate of the cell collection fluid is modified to follow a first profile, and wherein the first profile increases the first flow rate to a second flow rate over a second predetermined time period.

15. The method of claim 12, wherein the concentration of the cells within each of the plurality of groups of cells is increased by maintaining the cell collection fluid at the second flow rate for a third predetermined time period.

16. An aseptically closed method for volume reduction and washing of a cell suspension using counter-flow centrifugation, wherein the final cell composition comprises:
a final cell concentration of at least 20 million/ml;
a less than five percent drop in cell viability;
a product recovery of greater than 85 percent;
less than one microgram/ml of bovine serum albumin; and
less than one microgram/ml of enzymatically active harvest reagent.

17. The method of claim 1, wherein said cells produce less than 2-fold decrease in ATP production.

18. The method of claim 1, wherein said cells produce no more than 20 mU/ml Lactate Dehydrogensase per hr per 10⁶ cells or less than 3-fold increase in other shear-induced molecule release.

19. The method of claim 1, wherein the total number of viable cells in said composition is 2 billion to 30 billion.

20. The method of claim 1, wherein said cells are in a final harvest volume between 75 milliliter to 1200 milliliter.

21. The method of claim 3, wherein there is a dump volume of 30-40 milliliter.

22. The method of claim 1, wherein the first profile comprises operating parameters for a dynamic flow rate operation to follow a predetermined increase in net force for cell retention, wherein said operating parameters comprises:
the cell collection fluid first flow rate of between 30-50 ml/min;
the cell collection fluid second flow rate of between 120-160 ml/min;
a rate of increase in the first flow rate to the second flow of between 1-2 ml/min;
a centrifugal force value of between 500-1000 rcf; and
a wash flow rate of between 100-160 ml/min.

23. The method of claim 8, wherein the processing time is reduced by at least 0.5 hr.

24. The method of claim 8, wherein the processing time is reduced by at least 1 hour.

25. The method of claim 8, wherein the processing time is reduced by at least 2 hour.

26. The method of claim 1, further comprising the step of maintaining the cell collection chamber at less than 28° C.

27. The method of claim 11, wherein said product cells are substantially free of mycoplasma, endotoxin and microbial contamination.

28. The method of claim 11, wherein the time of cell transfer for further formulation and freeze step is less than 2 hours.

29. The method of claim 11, wherein the time of cell transfer for further formulation and freeze step is less than 1 hour.

30. The method of claim 1, wherein said product cells are in pharmaceutical-grade solution suitable for human administration.

31. The method of claim 11, wherein further automation is adapted to the collection step with controls using:
a viable cell density sensor means, for monitoring harvest density;
a flow meter monitoring means, for monitoring harvest volume;
a control logic software means, to control or achieve the desired final concentration of product cells in collection bag, and for operating valves, wherein said control logic software means are pneumatically or electrically activated and wherein the flow of fluid can be diverted from one collection bag to the other.

32. The aseptically closed method of claim 16, wherein the final cell composition is at least 30 million/ml.

33. The aseptically closed method of claim 16, wherein the final cell composition is at least 40 million/ml.

34. The aseptically closed method of claim 16, wherein the final cell composition is at least 50 million/ml.

35. The aseptically closed method of claim 16, wherein the drop in cell viability is less than three percent.

36. The aseptically closed method of claim 17, wherein the drop in cell viability is less than one percent.

37. The aseptically closed method of claim 17, wherein the product recovery is greater than 90 percent.

38. The aseptically closed method of claim 17, wherein the product recovery is greater than 95 percent.

39. The method of claim 1 wherein milliliter harvest volume is determined using the relationship y=37.865x+10 for product concentration of 20 million/ml, wherein x is total viable cells.

40. The method of claim 11, wherein said product cells are in pharmaceutical-grade solution suitable for human administration.
